(19) United States
(12) Patent Application Publication
(10) Pub. No.: US 2018/0271469 A1
(43) Pub. Date: Sep. 27, 2018

(54) RADIOGRAPHIC MOVING IMAGE PROCESSING APPARATUS

(71) Applicant: Konica Minolta, Inc., Tokyo (JP)

(72) Inventor: Yuki MATSUMOTO, Osaka (JP)

(21) Appl. No.: 15/869,958

(22) Filed: Jan. 12, 2018

(30) Foreign Application Priority Data
Mar. 22, 2017 (JP) 2017-055553

(51) Int. Cl. A61B 6/00; G06T 7/00; G06T 7/11

(52) U.S. Cl. CPC A61B 6/5217; G06T 7/0016; G06T 7/11; A61B 5/08; G06T 2207/10116; G06T 2207/30061; A61B 6/50

(57) ABSTRACT

A radiographic moving image processing apparatus includes: a detector that detects a non-pulmonary region included in each frame image among a plurality of frame images included in a radiographic moving image; a setter that sets a region of interest in each frame image, the region of interest including a plurality of pixels; and a calculator that performs a calculation to obtain a statistic from at least part of a plurality of pixel values of the plurality of pixels, and, in the calculation, performs exception processing on pixel values of pixels belonging to the non-pulmonary region among the plurality of pixels in the region of interest, the exception processing being different from processing performed on pixel values of pixels belonging to a pulmonary region among the plurality of pixels in the region of interest.

The entire disclosure of Japanese patent Application No. 2017-055553, filed on Mar. 22, 2017, is incorporated herein by reference in its entirety.

### BACKGROUND

### Technological Field

The present invention relates to a radiographic moving image processing apparatus.

### Description of the Related Art

A chest radiographic moving image includes a pulmonary region. Therefore, a chest radiographic moving image is used in dynamic analysis of the pulmonary field, such as ventilation analysis or analysis of the bloodstream in the pulmonary field. The technology disclosed in JP 4404291 B2 is an example of such use of an image. In the technology disclosed in JP 4404291 B2, an inter-frame difference image of respiratory dynamic images is created, and the created difference image is used in determining whether there is a disease (paragraph [0011] in JP 4404291 B2).

However, a chest radiographic moving image includes not only the pulmonary region but also non-pulmonary regions such as a costal region overlapping the pulmonary region and a fatty region adjacent to the pulmonary region. Therefore, in a conventional technology such as the technology disclosed in JP 4404291 B2, the pixels to be used in dynamic analysis of the pulmonary field include pixels belonging to the non-pulmonary regions. As a result, regions other than the pulmonary field might affect the dynamic analysis of the pulmonary field, and the dynamic analysis of the pulmonary field might not be conducted in an appropriate manner.

### SUMMARY

The present invention described below aims to solve the problem. An object of the present invention is to suppress the influence of the regions other than the pulmonary field on dynamic analysis of the pulmonary field and appropriately conduct the dynamic analysis of the pulmonary field in processing of a radiographic moving image.

To achieve the abovementioned object, according to an aspect of the present invention, a radiographic moving image processing apparatus reflecting one aspect of the present invention comprises: a detector that detects a non-pulmonary region included in each frame image among a plurality of frame images included in a radiographic moving image; a setter that sets a region of interest in each frame image, the region of interest including a plurality of pixels; and a calculator that performs a calculation to obtain a statistic from at least part of a plurality of pixel values of the plurality of pixels, and, in the calculation, performs exception processing on pixel values of pixels belonging to the non-pulmonary region among the plurality of pixels in the region of interest, the exception processing being different from processing performed on pixel values of pixels belonging to a pulmonary region among the plurality of pixels in the region of interest.

### BRIEF DESCRIPTION OF THE DRAWINGS

The objects, advantages, aspects, and features provided by one or more embodiments of the invention will become more fully understood from the detailed description given hereinbelow and the appended drawings which are given by way of illustration only, and thus are not intended as a definition of the limits of the present invention:

FIG. 1 is a block diagram showing a radiographic moving image capturing/processing system according to a first embodiment;

FIG. 2 is a diagram schematically showing a radiographic moving image generated in the radiographic moving image capturing/processing system according to the first embodiment;

FIG. 3 is a flowchart showing the flow of processing in the radiographic moving image capturing/processing system according to the first embodiment;

FIG. 4 is a graph showing an example of temporal changes in the average density values in a case where an exclusion process is performed and in a case where an exclusion process is not performed in the radiographic moving image capturing/processing system according to the first embodiment; and

FIG. 5 is a graph showing an example of temporal changes in the reference frame differences in a case where an exclusion process is performed and in a case where an exclusion process is not performed in the radiographic moving image capturing/processing system according to the first embodiment.

### DETAILED DESCRIPTION OF EMBODIMENTS

Hereinafter, one or more embodiments of the present invention will be described with reference to the drawings. However, the scope of the invention is not limited to the disclosed embodiments.

1. Radiographic Moving Image Capturing/Processing System

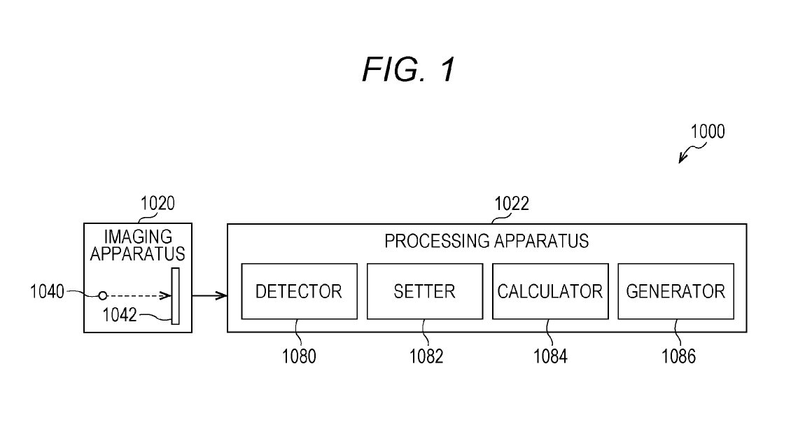

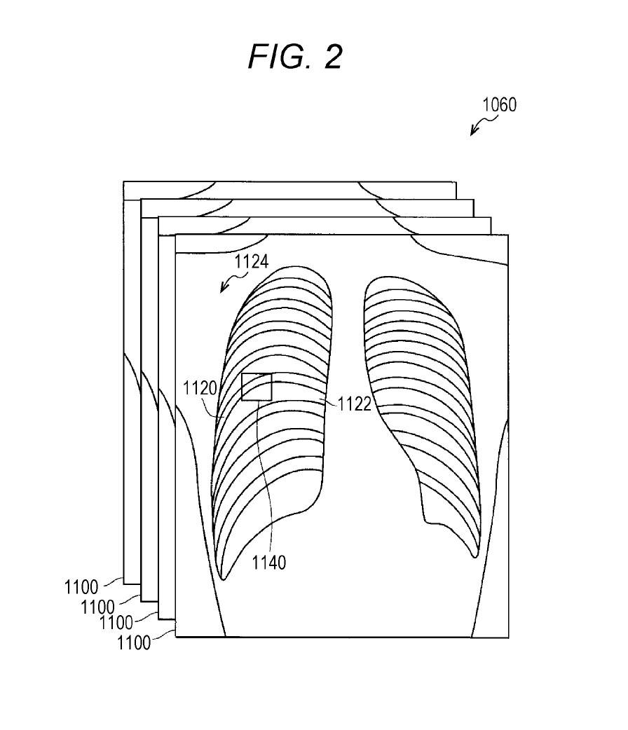

FIG. 1 is a block diagram showing a radiographic moving image capturing/processing system according to a first embodiment. FIG. 2 is a diagram schematically showing a radiographic moving image generated in the radiographic moving image capturing/processing system according to the first embodiment.

The radiographic moving image capturing/processing system 1000 shown in FIG. 1 includes an imaging apparatus 1020 and a processing apparatus 1022.

The imaging apparatus 1020 includes an X-ray source 1040 and a flat X-ray detector (a flat panel detector (FPD)) 1042, and generates a radiographic moving image 1060 shown in FIG. 2.

In one radiographic imaging operation, the imaging apparatus 1020 generates X-rays from the X-ray source 1040, causes the generated X-rays to penetrate through the human body, and detects the X-rays having penetrated through the human body with the FPD 1042. By doing so, the imaging apparatus 1020 generates a frame image including images of various anatomical regions in the body in the one radiographic imaging operation. By performing radiographic imaging more than once, the imaging apparatus 1020 generates the radiographic moving image 1060 including more than one frame image.

The radiographic moving image 1060 is also called a radiographic dynamic image. The radiographic moving image capturing/processing system 1000 is designed to radiographically captures the chest, and generate the chest radiographic moving image 1060. The chest radiographic moving image 1060 is to be used in dynamic analysis of the pulmonary field, such as ventilation analysis or analysis of the bloodstream in the pulmonary field.

The processing apparatus 1022 includes a detector 1080, a setter 1082, a calculator 1084, and a generator 1086, and processes the generated radiographic moving image 1060.

The detector 1080 detects the costal region 1120 included in each frame image 1100 of the frame images. Instead of the costal region 1120, a non-pulmonary region other than the costal region 1120 may be detected. For example, instead of the costal region 1120 overlapping a pulmonary region 1122, a fatty region 1124 adjacent to the pulmonary region 1122 may be detected.

The setter 1082 sets a region of interest (ROI) 1140 in each frame image 1100. The ROI 1140 includes pixels.

The calculator 1084 performs calculation to obtain a statistic in the ROI from at least one of the pixel values of the pixels included in the ROI 1140 in each frame image 1100. The statistic in the ROI is the maximum value, the minimum value, the average value, or the intermediate value of at least one of the pixel values. The statistic in the ROI may be a value other than these values.

In the calculation of the statistic in the ROI, regular processing is performed on the pixel values of the pixels belonging to the pulmonary region 1122 among the pixels included in the ROI 1140, and exception processing, which differs from the regular processing, is performed on the pixel values of the pixels belonging to the costal region 1120 among the pixels included in the ROI 1140.

A first example of the exception processing is a process in which the pixel values of the pixels belonging to the costal region 1120 are ignored. Hereinafter, the process in which the pixel values of the pixels belonging to the costal region 1120 will be also referred to as the exclusion process.

In a case where the calculated statistic in the ROI is the maximum value, the minimum value, or the intermediate value, and exception processing to ignore the respective pixel values b1, b2, . . . , and bn of the n pixels belonging to the costal region 1120 is to be performed, the maximum value, the minimum value, or the intermediate value is selected from among the respective pixel values a1, a2, . . . , and am of the m pixels belonging to the pulmonary region 1122.

In a case where the calculated statistic in the ROI is the average value, and exception processing to ignore the respective pixel values b1, b2, . . . , and bn of the n pixels belonging to the costal region 1120 is to be performed, the sum a1+a2+ . . . +am of the pixel values a1, a2, . . . , and am of the m pixels belonging to the pulmonary region 1122 is divided by the number m of the pixels belonging to the pulmonary region 1122, and thus, the average value (a1+a2+ . . . +am)/m is obtained.

In the first example of the exception processing, the respective pixel values b1, b2, . . . , and bn of the n pixels belonging to the costal region 1120 do not affect the statistic in the ROI. Thus, the influence of the regions other than the pulmonary field on the dynamic analysis of the pulmonary field is suppressed, and the dynamic analysis of the pulmonary field can be appropriately conducted.

A second example of the exception processing is a process in which a coefficient k is used for the respective pixel values b1, b2, . . . , and bn of the n pixels belonging to the costal region 1120. Using the coefficient k for the pixel values b1, b2, . . . , and bn is multiplying the pixel values b1, b2, . . . , and bn by the coefficient k, dividing the pixel values b1, b2, . . . , and bn by the coefficient k, adding the coefficient k to the pixel values b1, b2, . . . , and bn, subtracting the coefficient k from the pixel values b1, b2, . . . , and bn, or the like. The second example is preferably adopted in a case where the condition of the pulmonary field is reflected by the costal region 1120 to some extent.

In a case where the calculated statistic in the ROI is the maximum value, the minimum value, or the intermediate value, and exception processing to multiply the respective pixel values b1, b2, . . . , and bn of the n pixels belonging to the costal region 1120 by the coefficient k, the maximum value, the minimum value, or the intermediate value is selected from among the respective pixel values a1, a2, . . . , and am of the m pixels belonging to the pulmonary region 1122, and pixel values k·b1, k·b2, . . . , and k·bn obtained by multiplying the respective pixel values b1, b2, . . . , and bn of the n pixels belonging to the costal region 1120 by the coefficient k.

In a case where the calculated statistic in the ROI is the average value, and exception processing to multiply the respective pixel values b1, b2, . . . , and bn of the n pixels belonging to the costal region 1120 by the coefficient k, the sum a1+a2+ . . . +am+k·b1+k·b2+ . . . +k·bn of the respective pixel values a1, a2, . . . , and am of the m pixels belonging to the pulmonary region 1122 and pixel values k·b1, k·b2, . . . , and k·bn obtained by multiplying the respective pixel values b1, b2, . . . , and bn of the n pixels belonging to the costal region 1120 by the coefficient k is divided by the sum m+n of the number m of the pixels belonging to the pulmonary region 1122 and the number n of the pixels belonging to the costal region 1120, and thus, the average value (a1+a2+ . . . +am+k·b1+k·b2+. . . +k·bn)/(m+n) is obtained.

In the second example of the exception processing, the influence of the respective pixel values b1, b2, . . . , and bn of the n pixels belonging to the costal region 1120 on the statistic in the ROI can be made smaller than the influence of the respective pixel values a1, a2, . . . , and am of the m pixels belonging to the pulmonary region 1122 on the statistics in the ROI. Thus, the influence of the regions other than the pulmonary field on the dynamic analysis of the pulmonary field is suppressed, and the dynamic analysis of the pulmonary field can be appropriately conducted.

The generator 1086 calculates the difference between the statistic in the ROI obtained from a first frame image of the frame images and the statistic in the ROI obtained from a second frame image of the frame images, and generates a difference image between the first frame image and the second frame image. Alternatively, analytic information other than the difference image between the first frame image and the second frame image may be generated. For example, a graph showing temporal changes in the statistic in the ROI may be generated.

2. Processing Flow

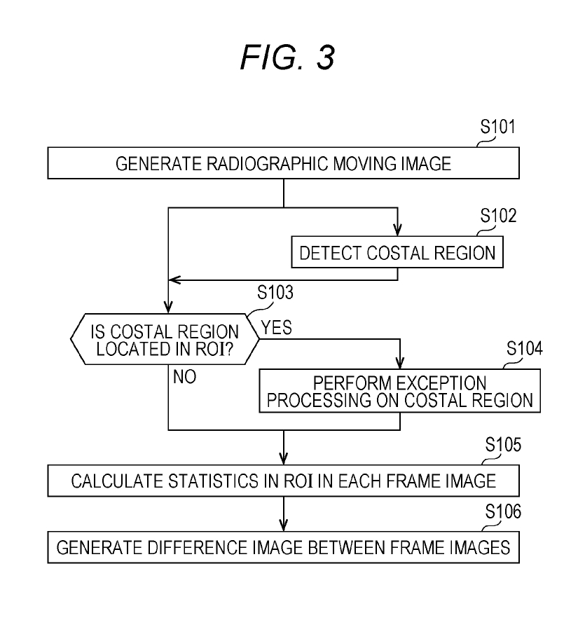

FIG. 3 is a flowchart showing the flow of processing in the radiographic moving image capturing/processing system according to the first embodiment.

In step S101 shown in FIG. 3, the imaging apparatus 1020 generates the radiographic moving image 1060.

In a case where the costal region 1120 is included in each frame image 1100, the detector 1080 detects the costal region 1120 included in each frame image 1100 in step S102, and the setter 1082 sets the ROI 1140 in each frame image 1100 and determines whether the costal region 1120 is located in the set ROI 1140 in step S103.

In a case where the costal region 1120 is not included in each frame image 1100, on the other hand, the detector 1080 does not detect any costal region 1120 included in each frame image 1100, and the setter 1082 sets the ROI 1140 in each frame image 1100 and determines whether the costal region 1120 is located in the set ROI 1140 in step S103.

In a case where the setter 1082 determines that the costal region 1120 is located in the ROI 1140, the calculator 1084 performs exception processing on the pixel values of the pixels belonging to the costal region 1120 in step S104, and the calculator 1084 calculates the statistic in the ROI 1140 for each frame image 1100 in step S105.

In a case where the setter 1082 determines that the costal region 1120 is not located in the ROI 1140, on the other hand, the calculator 1084 does not perform exception processing on the pixel values of the pixels belonging to the costal region 1120, and the calculator 1084 calculates the statistic in the ROI 1140 for each frame image 1100 in step S105.

In step S106, the generator 1086 generates a difference image between the frame images.

3. Differences to be Caused by an Exclusion Process

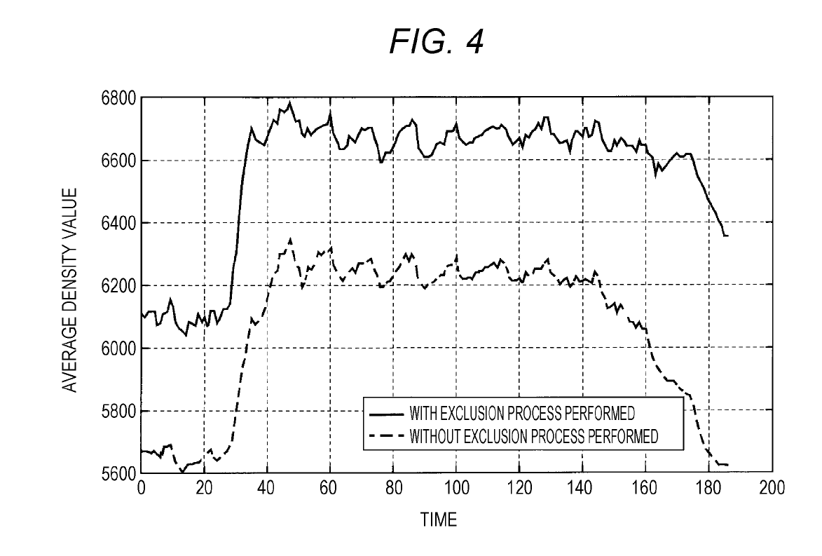

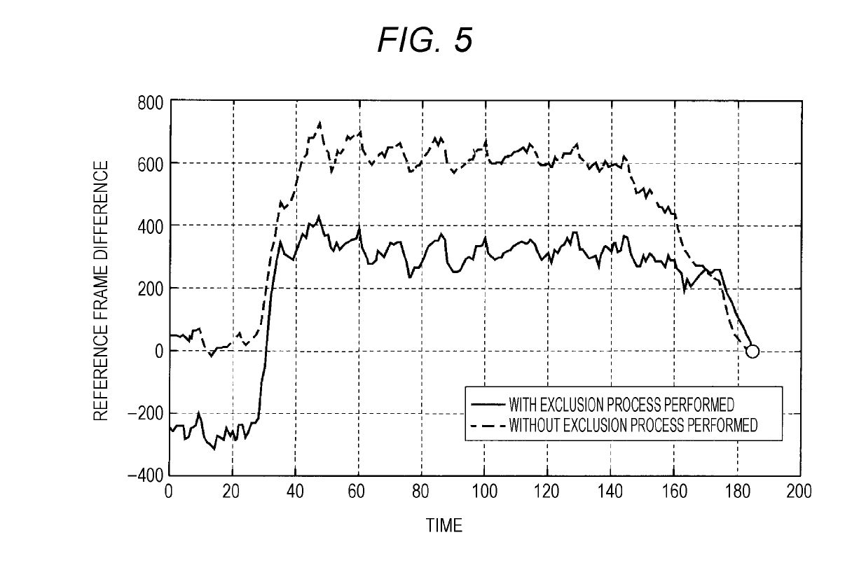

FIG. 4 is a graph showing an example of temporal changes in the average density values in a case where an exclusion process is performed and in a case where an exclusion process is not performed in the radiographic moving image capturing/processing system according to the first embodiment. FIG. 5 is a graph showing an example of temporal changes in the reference frame differences in a case where an exclusion process is performed and in a case where an exclusion process is not performed in the radiographic moving image capturing/processing system according to the first embodiment.

The pixel values of the pixels belonging to the costal region 1120 are normally lower than the pixel values of the pixels belonging to the pulmonary region 1122. Therefore, as shown in FIG. 4, the average density value in a case where an exclusion process is not performed is affected by the pixel values of the pixels belonging to the costal region 1120, and is lower than the average density value in a case where an exclusion process is performed. Because of this, the reference frame difference in a case where an exclusion process is not performed might become larger than the reference frame difference in a case where an exclusion process is performed, as shown in FIG. 5. In some circumstances, the reference frame difference in a case where an exclusion process is not performed might become smaller than the reference frame difference in a case where an exclusion process is performed. That is, in a case where an exclusion process is performed, a highly accurate average density value and a highly accurate reference frame difference are obtained. In a case where an exclusion process is not performed, however, a highly accurate average density value and a highly accurate reference frame difference are not obtained.

Although embodiments of the present invention have been described and illustrated in detail, the disclosed embodiments are made for purposes of illustration and example only and not limitation. The scope of the present invention should be interpreted by terms of the appended claims. It should be understood that numerous modifications not mentioned herein can be made without departing from the scope of the invention.

What is claimed is:

1. A radiographic moving image processing apparatus comprising:
a detector that detects a non-pulmonary region included in each frame image among a plurality of frame images included in a radiographic moving image;
a setter that sets a region of interest in each frame image, the region of interest including a plurality of pixels; and
a calculator that performs a calculation to obtain a statistic from at least part of a plurality of pixel values of the plurality of pixels, and, in the calculation, performs exception processing on pixel values of pixels belonging to the non-pulmonary region among the plurality of pixels in the region of interest, the exception processing being different from processing performed on pixel values of pixels belonging to a pulmonary region among the plurality of pixels in the region of interest.

2. The radiographic moving image processing apparatus according to claim 1,
wherein the non-pulmonary region is a costal region.

3. The radiographic moving image processing apparatus according to claim 1,
wherein, in the exception processing, statistic processing is performed, with a density change in the pulmonary region and a density change in the non-pulmonary region being distinguished from each other.

4. The radiographic moving image processing apparatus according to claim 3,
wherein the exception processing is a process to ignore the pixel values of the pixels belonging to the non-pulmonary region.

5. The radiographic moving image processing apparatus according to claim 1,
wherein the exception processing is a process to apply a coefficient to the pixel values of the pixels belonging to the non-pulmonary region.

6. The radiographic moving image processing apparatus according to claim 1, further comprising
a generator that calculates a difference between a statistic value obtained from a first frame image included in the plurality of frame images and a statistic value obtained from a second frame image included in the plurality of frame images, and generates a difference image between the first frame image and the second frame image.
